(19) United States
(12) Patent Application Publication
(10) Pub. No.: US 2008/0095757 A1
(43) Pub. Date: Apr. 24, 2008

(54) VITAMIN C COMPOSITIONS

(75) Inventor: NEIL LEVIN, St. Charles, IL (US)

Correspondence Address: PATENT GROUP;C/O DLA PIPER US LLP, 203 N. LASALLE ST., SUITE 1900, CHICAGO, IL, 60601, US

(73) Assignee: NOW HEALTH GROUP, INC.

(21) Appl. No.: 11/877,230

(22) Filed: Oct. 23, 2007

Related U.S. Application Data
(60) Provisional application No. 60/853,803, filed on Oct. 23, 2006.
(60) Provisional application No. 60/878,123, filed on Jan. 3, 2007.

(51) Int. Cl. A61K 31/375; A61K 31/355; A61K 31/70; A61K 33/04; A61K 33/30; A61K 33/34; A61K 36/00; A61K 36/87; A61K 38/48; A61P 39/06

(52) U.S. Cl. 424/94.63; 424/630; 424/641; 424/702; 424/725; 424/736; 424/737; 424/777; 424/94.1; 514/25; 514/458; 514/474

(57) ABSTRACT

The field of invention relates to Vitamin C compositions, and in particular to Vitamin C compositions containing ascorbate-glucose transport enhancers. In at least one aspect, a composition is provided herein that includes ascorbate in an amount from about 0.1% by weight of actives to about 99.9% by weight of actives, and at least one ascorbate-glucose transport enhancer in an amount from about 0.01% by weight of actives to about 99.0% by weight of actives. In another aspect, a method of improving the transport of ascorbate into cells and tissues is provided that includes providing a composition comprising ascorbate and at least one ascorbate-glucose transport enhancer.

### RELATED APPLICATIONS

This application claims the benefit of U.S. Provisional Application Ser. No. 60/853,803, filed Oct. 23, 2006, now pending, and also claims the benefit of U.S. Provisional Application Ser. No. 60/878,123, filed Jan. 3, 2007, now pending. The disclosures of U.S. Provisional Application Ser. Nos. 60/853,803 and 60/878,123 are hereby incorporated by reference in their entirety.

### BACKGROUND

The field of invention relates to Vitamin C compositions, and in particular to Vitamin C compositions containing ascorbate-glucose transport enhancers.

Ascorbate, also referred to as vitamin C or ascorbic acid, is an important nutrient for humans. Ascorbate is often in the form of L-ascorbic acid, and can also be in other forms, such as, for example, L-xylo-ascorbic acid, or L-threo-hex-2-enoic acid γ-lactone. Ascorbate is known as an antioxidant because it is an electron donor, and is thus a reducing agent. “[B]y donating its electrons, it prevents other compounds from being oxidized. However, by the very nature of this reaction, vitamin C itself is oxidized in the process.” Padayatty S J, et al., “Vitamin C as an antioxidant: evaluation of its role in disease prevention,” J Am Coll Nutr. February 2003; 22(1):18-35, 19.

According to a review study by NIH researchers, “Vitamin C in humans must be ingested for survival. Vitamin C is an electron donor, and this property accounts for all its known functions. As an electron donor, vitamin C is a potent water-soluble antioxidant in humans. Antioxidant effects of vitamin C have been demonstrated in many experiments in vitro. Human diseases such as atherosclerosis and cancer might occur in part from oxidant damage to tissues.” Padayatty S J, et al., “Vitamin C as an antioxidant: evaluation of its role in disease prevention,” J Am Coll Nutr. February 2003; 22(1):18-35. Further, “lack of dietary ascorbate results in the clinical syndrome scurvy.” Rumsy et al., “Absorption, Transport, and Disposition of Ascorbic Acid in humans,” Nutritional Biochemistry 9:116-130, 116 (1998). Nevertheless, “[D]espite [ ] data indicating a small increase in the median dietary vitamin C ingestion in the USA, a substantial fraction of the population still ingests vitamin C at or below the Recommended Dietary Allowance.” Padayatty S J, et al., “Vitamin C as an antioxidant: evaluation of its role in disease prevention,” J Am Coll Nutr. February 2003; 22(1):18-35, 22.

It has been demonstrated that vitamin C works more effectively as an antioxidant in the presence of lipoic acid compounds. For example, “[t]he presence of DHLA in the reaction mixture containing ascorbate extended the recycling reaction through regeneration of ascorbate.” Kagan V E, et al., “Direct evidence for recycling of myeloperoxidase-catalyzed phenoxyl radicals of a vitamin E homologue, 2,2,5,7,8-pentamethyl-6-hydroxy chromane, by ascorbate/dihydrolipoate in living HL-60 cells,” Biochim Biophys Acta. Mar. 17, 2003;1620(1-3):72-84. Further, “[t]he water-soluble antioxidant vitamin C can reduce tocopheroxyl radicals directly or indirectly and thus support the antioxidant activity of vitamin E; such functions can be performed also by other appropriate reducing compounds such as glutathione (GSH) or dihydrolipoate.” Sies H, et al., “Antioxidant functions of vitamins. Vitamins E and C, beta-carotene, and other carotenoids,” Ann N Y Acad Sci. Sep. 30, 1992;669:7-20. Review.

Studies have also shown that lipoic acid isomers and metabolites can affect glucose transport mechanisms and insulin sensitivity. “The effect of alpha-lipoate and dihydrolipoate on the mitochondrial permeability transition was investigated. Both substances promoted the permeability transition in isolated rat liver mitochondria and in permeabilized hepatocytes, dihydrolipoate most potently in spite of it being a dithiol. The stimulation was prevented by Cyclosporin A or hydroxybutyltoluene but not by ascorbate.” Saris N E, et al., “The stimulation of the mitochondrial permeability transition by dihydrolipoate and alpha-lipoate,” Biochem Mol Biol Int. January 1998;44(1):127-34. “LA treatment prevented this reduction, resulting in insulin-stimulated glucose uptake comparable to that of nondiabetic animals. These results suggest that daily LA treatment may reduce blood glucose concentrations in STZ-diabetic rats by enhancing muscle GLUT4 protein content and by increasing muscle glucose utilization.” Khamaisi M, et al., “Lipoic acid reduces glycemia and increases muscle GLUT4 content in streptozotocin-diabetic rats,” Metabolism. July 1997;46(7):763-8. PMID: 9225829. “As can be expected, administration of antioxidants such as lipoic acid in oxidized cells, in animal models of diabetes, and in type 2 diabetes shows improved insulin sensitivity. Thus, oxidative stress is presently accepted as a likely causative factor in the development of insulin resistance.” Bloch-Damti A, Bashan N., “Proposed mechanisms for the induction of insulin resistance by oxidative stress,” Antioxid Redox Signal. November-December 2005; 7(11-12):1553-67. Review. PMID: 16356119. “Alpha-Lipoic acid was recently shown to stimulate glucose uptake into 3T3-L1 adipocytes by increasing intracellular oxidant levels and/or facilitating insulin receptor autophosphorylation presumably by oxidation of critical thiol groups present in the insulin receptor beta-subunit.” Moini H, Packer L, Saris N E., “Antioxidant and prooxidant activities of alpha-lipoic acid and dihydrolipoic acid,” Toxicol Appl Pharmacol. Jul. 1, 2002;182(1):84-90. Review. PMID: 12127266.

Additionally, lipoic acid can affect the ascorbate-GSH antioxidant system. “The influence of alpha-lipoic acid (CAS 62-46-4) on the amount of intracellular glutathione (GSH) was investigated in vitro and in vivo. Using murine neuroblastoma as well as melanoma cell lines in vitro, a dose-dependent increase of GSH content was observed. Dependent on the source of tumor cells the increase was 30-70% compared to untreated controls. Normal lung tissue of mice also revealed about 50% increase in glutathione upon treatment with lipoic acid. This corresponds with protection from irradiation damage in these in vitro studies.” Busse E, et al., “Influence of alpha-lipoic acid on intracellular glutathione in vitro and in vivo,” Arzneimittelforschung. June 1992; 42(6):829-31.

The text of each of the above cited references is hereby incorporated by reference in its entirety.

### BRIEF SUMMARY

The compositions disclosed herein are compositions containing acorbate. More specifically, the compositions disclosed herein comprise ascorbate and at least one ascorbate-glucose transport enhancer. Although not being bound by any particular theory, it is believed that such compositions can improve the cellular uptake of ascorbate.

In at least one aspect, a composition is provided herein that includes ascorbate in an amount from about 0.1% by weight of actives to about 99.9% by weight of actives, and at least one ascorbate-glucose transport enhancer in an amount from about 0.01% by weight of actives to about 99.0% by weight of actives. It is preferred that the ascorbate be in the form of vitamin C, ascorbic acid, L-ascorbic acid, an ascorbyl ester, ascorbyl palmitate, an ascorbyl phosphate ester, a reacted or blended mineral ascorbate, dehydroascorbate (also known as DHA, DHAA, and oxidized vitamin C), or a vitamin C metabolite. The ascorbate can be provided by one source, or by a plurality of sources. Further, it is also preferred that the at least one ascorbate-glucose transport enhancer be lipoic acid or corosolic acid.

In at least another aspect, methods of improving the transport of acorbate into cells and tissues are provided herein. Such methods include providing a composition comprising ascorbate and at least one ascorbate-glucose transport enhancer. The composition can be in any suitable form, but is preferably in an oral dosage form or a topical dosage form. It is particularly preferred that the ascorbate is in an amount from about 0.1% by weight of actives to about 99.9% by weight of actives. It is also preferred that the at least one ascorbate-glucose transport enhancer be present in an amount from about 0.01% by weight of actives to about 99.0% by weight of actives.

### DETAILED DESCRIPTION

Studies have shown that a significant number of Americans do not consume sufficient amounts of ascorbate in their daily diet, such as by consuming adequate servings of fruits and vegetables. Increasing the amount of ascorbate consumed in a standard daily diet is one way to rectify this deficiency. Dietary supplements are another option, and there are a number of commercially available vitamin C supplements. The effectiveness of such supplements is limited, however, by the absorption rates and transport efficiencies of the body.

Individuals with impaired glucose mechanisms, including diabetes and metabolic syndrome, may have cellular insulin resistance that also impairs ascorbate transport. Such an impairment could result in a functional vitamin C deficiency at the cellular level even if dietary sources seem adequate. One consequence is that individuals with impaired glucose metabolism may have complications that arise from overproduction of reactive oxygen and nitrogen, which the body could maintain within normal limits if it had access to adequate dietary and cellular intake of ascorbate and other appropriate antioxidant nutrients.

Compositions disclosed herein comprise ascorbate and at least one ascorbate-glucose transport enhancer. Such compositions may be useful in improving a person's ascorbate status. For example, such compositions may improve ascorbate and antioxidant status for diabetics and other people with cellular insulin resistance.

It has been found that such compositions provide a synergistic effect with respect to the transport and/or recycling of ascorbate within the human body. While not being bound by any particular theory, it is believed that ascorbate-glucose transport enhancers improve the transport of ascorbate into cells and tissues primarily by utilizing the glucose transport system. It is also believed that some ascorbate-glucose transport enhancers may enhance ascorbate transfer by increasing other antioxidant stores, including those in the glutathione family. It is further believed, that the present compositions may decrease ROS (reactive oxygen species) activities and improve nitric oxide distribution.

Accordingly, the present technology provides methods of improving the transport of ascorbate into cells and tissues. Such methods include providing a composition comprising ascorbate and at least one ascorbate-glucose transport enhancer. The composition can be administered to a person in any way that results in providing the composition to cells and/or tissues. A composition can be in any suitable form for such administration, such as, for example, an oral dosage form or a topical dosage form. The compositions suitable for use with this method of improving the cellular uptake of ascorbate are discussed below.

### Ascorbate

Ascorbate for use in the present compositions can be in any suitable form. For example, ascorbate can be in the form of vitamin C, ascorbic acid, L-ascorbic acid, L-xylo-ascorbic acid, L-threo-hex-2-enoic acid γ-lactone, an ascorbyl ester, ascorbyl palmitate, an ascorbyl phosphate ester, a reacted or blended mineral ascorbate, dehydroascorbate (also known as DHA, DHAA, and oxidized vitamin C), a vitamin C metabolite, a derivative thereof, or an equivalent thereof. Ascorbyl phosphate esters can include, but are not limited to mono, di, and tri sodium phosphates, magnesium phosphates, and calcium salt phosphates. Ascorbate can be present in a composition in a single form, or in multiple forms.

Mineral ascorbates are compounds of minerals and vitamin C that are typically reacted together, but can also be provided as an unreacted blend of ingredients. Examples of mineral ascorbates include, for example, calcium ascorbate, magnesium ascorbate, zinc ascorbate, sodium ascorbate, and potassium ascorbate.

Ascorbate in the present compositions can be provided by a single source, or can be provided by multiple sources. For example, ascorbate can be provided by any natural or synthesized source. Natural sources include, for example, fruits and vegetables. Some fruit sources rich in ascorbate include, for example, cantaloupe, grapefruit, honeydew, kiwi, mango, orange, papaya, strawberries, tangelo, tangerine, and watermelon. Some vegetable sources rich in ascorbate include, for example, asparagus, broccoli, brussels sprouts, cabbage, cauliflower, kale, mustard greens, peppers (red or green), plantains, potatoes, snow peas, sweet potatoes, and tomatoes. In some particular compositions, the ascorbate is provided by at least one source selected from the group consisting of vegetables, fruit, camu fruit, alma berries, acerola cherries, rosehips, citrus fruit, extracts thereof, concentrates thereof, constituents thereof, or derivatives thereof.

In preferred embodiments, compositions include ascorbate in an amount from about 0.1% by weight of actives to about 99.9% by weight of actives. For example, a composition can include ascorbate in amounts of about 0.1%, about 0.2%, about 0.5%, about 1%, about 2%, about 5%, about 7%, about 10%, about 12%, about 15%, about 18%, about 20%, about 22%, about 24%, about 25%, about 27%, about 30%, about 32%, about 35%, about 37%, about 40%, about 42%, about 45%, about 47%, about 50%, about 52%, about 55%, about 57%, about 60%, about 62%, about 65%, about 67%, about 70%, about 72%, about 75%, about 77%, about 80%, about 82%, about 85%, about 87%, about 90%, about 92%, about 95%, about 97%, about 98%, about 99%, about 99.5%, or about 99.9% by weight of actives. Preferably, the ascorbate is present in amounts up to about 50% by weight of actives, or greater than about 50% by weight of actives. More preferably, the ascorbate is present in amounts up to about 80% by weight of actives, or greater than about 80% by weight of actives. Most preferably, the ascorbate is present in amounts up to about 90% by weight of actives, or greater than about 90% by weight of actives. For example, the ascorbate can be present in amounts from about 90% by weight of actives to about 99.9% by weight of actives, from about 95% by weight of actives to about 99.9% by weight of actives.

### Ascorbate-Glucose Transport Enhancers

Ascorbate-glucose transport enhancers for use in the present compositions include any substance that utilizes glucose transport mechanisms to improve cellular ascorbate transport. Ascorbate-glucose transport enhancers can be antioxidants, but are not necessarily antioxidants. For example, a particularly preferred ascorbate-glucose transport enhancer is lipoic acid. Lioic acid reduces (recharges) glutathione (GSH), an important antioxidant that is known to interact synergistically with vitamin C. Lipoic acid can be present in the present compositions in any suitable form, including alpha lipoic acid, ALA, r-alpha lipoic acid, RS-alpha lipoic acid, lipoate, as well as any equivalents thereof, derivatives thereof, related compounds or metabolites thereof. Other examples of preferred ascorbate-glucose transport enhancers include, but are not limited to, corosolic acid and its analogs, triterpenes with similar activity, such as, for example, Asiatic Acid and its analogs, as well as any equivalents thereof, derivatives thereof, related compounds, or metabolites thereof.

In preferred embodiments, compositions include at least one ascorbate-glucose transport enhancer, and can include a plurality of ascorbate-glucose transport enhancers. Preferably the at least one ascorbate-glucose transport enhancer is present in an amount from about 0.01% by weight of actives to about 99.0% by weight of actives. For example, a composition can include at least one ascorbate-glucose transport enhancer in amounts of about 0.01%, about 0.02%, about 0.05%, about 0.08%, about 0.1%, about 0.2%, about 0.3$ %, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 12%, about 15%, about 18%, about 20%, about 22%, about 24%, about 25%, about 27%, about 30%, about 32%, about 35%, about 37%, about 40%, about 42%, about 45%, about 47%, about 50%, about 52%, about 55%, about 57%, about 60%, about 62%, about 65%, about 67%, about 70%, about 72%, about 75%, about 77%, about 80%, about 82%, about 85%, about 87%, about 90%, about 92%, about 95%, about 97%, about 98%, about 98.5%, about 98.9%, or about 99% by weight of actives. Preferably, the at least one ascorbate-glucose transport enhancer is present in amounts up to about 5% by weight of actives, up to about 10% by weight of actives, or greater than about 10% by weight of actives. More preferably, the at least one ascorbate-glucose transport enhancer is present in amounts from about 0.01% by weight of actives to about 10% by weight of actives. Most preferably, the at least one ascorbate-glucose transport enhancer i s present in an amount from about 5% by weight of actives to about 10% of by weight of actives.

### Compositions

Compositions disclosed herein include ascorbate and at least one ascorbate-glucose transport enhancer. The preferred amounts of ascorbate and at least one ascorbate-glucose transport enhancer are discussed above. In at least one particularly preferred embodiment, a composition includes ascorbate in an amount from about 0.1% by weight of actives to about 99.9% by weight of actives and at least one ascorbate-glucose transport enhancer in an amount from about 0.01% by weight of actives to about 99.0% by weight of actives. The total weight of actives is determined by the total weight of all compositional components providing ascorbate and all compositional components acting as ascorbate-glucose transport enhancers. The total weight percentages of the ascorbate providing components and the ascorbate-glucose transport enhancer componenis of a composition should thus equal 100%.

Other components can also be present in the present compositions. The weight of a composition is the total weight of each of the components of the composition, not including any weight added by excipients.

For example, compositions can include antioxidants, amino acid compounds, and other components. For example, one preferred amino acid compound is threonic acid (also known as calcium threonate). In some embodiments, the present compositions can include from about 0.1% by weight of the composition to about 90.0% by weight of the composition of an antioxidant, a threonic acid, a fruit extract, a fruit concentrate, a vegetable extract, a vegetable concentrate, a mineral, a B-Vitamin, a B-vitamin metabolite. a Carotenoid, a CoQ10, a Grapeseed extract, a Green Tea, a Lutein, a Lycopene, a Pomegranate, a Pycnogenol, a Resveratrol, a Selenium, a Zeaxanthin, a Zinc, a Copper, a Vitamin E, a Tocopherol, or a Tocotrienol.

Compositions can also include other ingredients suitable for inclusion in a dietary supplement, such as, for example, nutritional co-factors for antioxidant nutrients and vitamins. For example, compositions can include from about 1% by weight of the composition to about 95% by weight of the composition of a pepper extract, a quercetin, a rutin, a bromelain, a polyphenol, or a bioflavonoid.

Compositions can further include at least one excipient. Excipients can include, but are not limited to magnesium stearate, a stearic acid, a microcrystalline cellulose, a calcium carbonate, a croscarmelose, silicon dioxide, or a starch.

### Product Forms

Compositions disclosed herein can be provided in any suitable dosage form. Preferably compositions are provided in an oral dosage form or a topical dosage form. For example, compositions can be in a dosage form that is a powder, a microencapsulated powder, granules, a granulated powder, a liquid, a gel, a lotion, a cream, a spray, an emulsion, an oil, an instant beverage, a liquid beverage, a beverage mix, a capsule, a softgel capsule, a two-piece capsule, a tablet, a chewable tablet, an effervescent tablet, a pre-blended mixture of ingredients, or a blended mixture of ingredients.

Compositions disclosed herein can also be provided in any suitable type of formulation. For example, compositions can be formulated as a time release formulation, a gradual release formulation, or a fast release formulation. As another example, compositions can also be formulated as an antioxidant vitamin formula, a multiple vitamin formula, an immune formula, or a joint formula.

Various embodiments of the compositions and methods of the present technology are detailed further in the following examples, which are provided for illustrative purposes and are not intended to limit the scope of the present invention.

### EXAMPLES

### Example 1

### Sample Formulations

The following compositions are examples of compositions of the present technology. The amounts of each of the components for Formulations 1-6 are stated in milligrams (mg). It should be noted that the formulations can contain any desired amount of excipients, and examples of preferred excipients are provided in each of the listed formulations. With respect to Formulations 7 and 8, the components are stated in terms of the amount of vitamin C provided, or the amount of ascorbate-glucose transport enhancer provided.

Formulation #1

| Component | Amount (mg) |
|---|---|
| Vitamin C | 500 |
| Alpha lipoic acid | 25 |
| Bioflavonoids | 150 |
| Acerola cherry | 50 |
| Rose hips | 50 |
| Rutin | 50 |
| Excipients (such as magnesium stearate) | |

Formulation #2

| Component | Amount (mg) |
|---|---|
| Vitamin C | 500 |
| Alpha lipoic acid | 50 |
| Bioflavonoids | 150 |
| Acerola cherry | 50 |
| Rose hips | 50 |
| Rutin | 50 |
| Excipients (such as magnesium stearate) | |

Formulation #3

| Component | Amount (mg) |
|---|---|
| Vitamin C | 500 |
| Alpha lipoic acid | 25 |
| Bioflavonoids | 200 |
| Acerola cherry | 75 |
| Rose hips | 75 |
| Rutin | 50 |
| Excipients (such as magnesium stearate, | |
| stearic acid, microcrystalline cellulose) | |

Formulation #4

| Component | Amount (mg) |
|---|---|
| Vitamin C | 500 |
| Alpha lipoic acid | 50 |
| Bioflavonoids | 200 |
| Acerola cherry | 75 |
| Rose hips | 75 |
| Rutin | 50 |
| Excipients (such as magnesium stearate, | |
| stearic acid, microcrystalline cellulose) | |

Formulation #5

| Component | Amount (mg) |
|---|---|
| Vitamin C | 1000 |
| Alpha lipoic acid | 100 |
| Bioflavonoids | 150 |
| Acerola cherry | 25 |
| Rose hips | 25 |
| Rutin | 25 |
| Excipients (such as magnesium stearate, | |
| stearic acid, microcrystalline cellulose, | |
| calcium carbonate, croscarmelose) | |

Formulation #6

| Component | Amount (mg) |
|---|---|
| Vitamin C | 1000 |
| Alpha lipoic acid | 50 |
| Bioflavonoids | 150 |
| Acerola cherry | 25 |
| Rose hips | 25 |
| Rutin | 25 |
| Excipients (such as magnesium stearate, | |
| stearic acid, microcrystalline cellulose, | |
| calcium carbonate, croscarmelose, silicon | |
| dioxide) | |

Formulation #7

| Component | Providing |
|---|---|
| Calcium Ascorbate | 220 mg Vitamin C |
| Magnesium Ascorbate | 220 mg Vitamin C |
| Potassium Ascorbate | 25 mg Vitamin C |
| Zinc Ascorbate | 10 mg Vitamin C |
| Ascorbyl Palmitate | 25 mg Vitamin C |
| Alpha lipoic acid | 25 mg ALA |

Formulation #8

| Component | Providing |
|---|---|
| Calcium Ascorbate | 220 mg Vitamin C |
| Magnesium Ascorbate | 220 mg Vitamin C |
| Potassium Ascorbate | 25 mg Vitamin C |
| Zinc Ascorbate | 10 mg Vitamin C |
| Ascorbyl Palmitate | 25 mg Vitamin C |
| Alpha lipoic acid | 50 mg ALA |

### Example 2

### Test Formulation A

Test Formulation A was produced by combining Formulation #8, as set forth in Example 1 above, with the other components listed below.

Test Formulation A

| Component | Amount (mg) |
|---|---|
| Formulation #8 | 1466.42 |
| Bioflavinoid Complex | 150.00 |
| Acerola Pure | 25.00 |
| Rose Hip Powder | 25.00 |
| Rutin | 25.00 |
| Vivapur 102 | 100.00 (Excipient) |
| Stearic Acid | 55.00 (Excipient) |
| Magnesium Stearate | 12.00 (Excipient) |
| Calcium Carbonate | 100.00 (Excipient) |
| Croscarmelose | 10.00 (Excipient) |

Human whole blood in vitro was exposed to (“spiked” with) either a Control (Vitamin C with Rose Hips), or to Test Formulation A. The spiking experiment was done and all results were obtained at the Sick Children's Hospital, Toronto, Ontario, Canada (Emadi-Konjin et al, 2005). The final concentration of Vitamin C used in the “spiking” solution was 1.0 mg/dL. This concentration of Vitamin C was chosen to represent about twice the rormal plasma level of Vitamin C (0.50 mg/dL).

The blood was sampled at Time Zero (immediately before adding the Control or Test Formulation A), after 30 minutes of exposure, and after 60 minutes of exposure. Each sample of whole blood was separated into a plasma fraction and a lymphocyte fraction for testing of Vitamin C concentration. The amount of Vitamin C (as ascorbic acid) in each fraction sample was determined by HPLC (Emadi-Konjin et al, 2005).

The testing procedure was performed as follows:

### Procedure:

a. Prepare. Test Solutions of the Control and Test Formulation A to add to whole blood samples so that the “spiking” solution is 10× the final concentration wanted in the final mixture.
b. At Time Zero, immediately before spiking, remove an aliquot of whole blood to test the plasma and lymphocyte fractions for initial Vitamin C concentration.
c. Add 1 part of the 10× solutions to 9 parts of whole blood to begin the timed exposure trials.
d. A plasma and a lymphocyte fraction are prepared from each of these 5 whole blood samples, giving a total of 10 samples for HPLC analysis:
   1. Whole blood at Time zero
   2. Whole blood plus 1.0 mg/dL Vitamin C at 30 minutes
   3. Whole blood plus 1.0 mg/dL Vitamin C at 60 minutes
   4. Whole blood plus 1.0 mg/dL Vitamin Cx at 30 minutes
   5. Whole blood plus 1.0 mg/dL Vitamin Cx at 60 minutes

Table A shows that, prior to spiking, the measured concentrations in the two spike stock solutions were slightly higher than the 10× target.

TABLE A
Pill Dilutions

| | | Measured |
|---|---|---|
| | Target Concentration | Concentration |
| Formulation | (mg/dL) | (mg/dL) |
| Control | 10.0 | 10.16 |
| Test Formulation A | 10.0 | 10.70 |

Table B gives the measured Vitamin C concentration in the plasma fraction. The reference range for fasting Vitamin C in plasma is 0.2 to 0.6 mg/dL (Jacob et al, 1987).

TABLE B
Plasma Fraction

| | | | Change in | % Change |
|---|---|---|---|---|
| | | | Vitamin C | in Vitamin |
| | | | from | C from |
| | | Measured | Baseline for | Baseline for |
| PLASMA | | Vitamin C | Plasma | Plasma |
| Formulation | Time | (mg/dL) | (mg/dL) | (mg/dL) |
| Blank | 0′ | 1.61 | NA | NA |
| Control | 30′ | 2.83 | 1.22 | 76% |
| Test Formulation A | 30′ | 2.91 | 1.30 | 81% |
| Control | 60′ | 2.68 | 1.07 | 66% |
| Test Formulation A | 60′ | 2.75 | 1.14 | 71% |

Table C gives the measured Vitamin C concentration in the lymphocyte fraction. The reference range for fasting Vitamin C in lymphocytes is about 10 to 25 ug/10⁸ lymphocytes (Jacob et al, 1987).

TABLE C
Lymphocyte Fraction

| | | | Change in | % Change |
|---|---|---|---|---|
| | | | Vitamin C | in Vitamin C |
| | | | from | from |
| | | Measured | Baseline for | Baseline for |
| LYMPHOCYTES | | Vitamin C | Cells (ug/10⁸ | Cells (ug/10⁸ |
| Formulation | Time | (ug/10⁸ cells) | cells) | cells) |
| Blank | 0′ | 15.3 | NA | NA |
| Control | 30′ | 35.2 | 19.9 | 130% |
| Test Formulation A | 30′ | 25.1 | 9.8 | 64% |
| Control | 60′ | 36.2 | 20.9 | 137% |
| Test Formulation A | 60′ | 16.4 | 1.1 | 7% |

The changes in Vitamin C levels in the plasma showed about the same percentage increases for the Control formulation and for Test Formulation A.

With respect to lymphocytes, the Control formulation showed an initial increase of 130% during the first 30 minutes, followed by an additional small increase over this amount from 30 to 60 minutes. These results are consistent with the hypothesis that the lymphocytes are equilibrating with the plasma level of Vitamin C surrounding them. Test Formulation A showed a 64% increase during the first 30 minutes, followed by a decrease to only 7% over the base amount at 60 minutes. These results indicate that the Vitamin C that was enhanced with ALA is being utilized by the lymphocytes over the time course of the trial. Since the amount of Vitamin C in the plasma sample enhanced with ALA also decreased during the trial, the Vitamin C is apparently not just leaking back into the plasma. If it is not leaking back into the plasma, it is most likely being utilized by the lymphocytes. Any utilization of the Vitamin C in the lymphocytes would stimulate further uptake of Vitamin C from the plasma into the lymphocytes. This utilization, associated with greater uptake of Vitamin C, occurs in the presence of ALA.

This experiment utilized lymphocytes as a model cell to study uptake and utilization kinetics of Vitamin C enhanced with ALA. The expectation is that other cell types will also show increased uptake and utilization of Vitamin C when it is made available with ALA.

From the foregoing, it will be appreciated that although specific embodiments of the invention have been described herein for purposes of illustration, various modifications may be made without deviating from the spirit or scope of the invention. It is therefore intended that the foregoing detailed description be regarded as illustrative rather than limiting, and that it be understood that it is the following claims, including all equivalent, that are intended to particularly point out and distinctly claim the subject matter regarded as the invention.

What is claimed is:

1. A composition comprising ascorbate and at least one ascorbate-glucose transport enhancer.

2. The composition of claim 1, wherein the ascorbate is in the form of vitamin C, ascorbic acid, L-ascorbic acid, L-xylo-ascorbic acid, L-threo-hex-2-enoic acid γ-lactone, an ascorbyl ester, ascorbyl palmitate, an ascorbyl phosphate ester, a reacted or blended mineral ascorbate, dehydroascorbate, or a vitamin C metabolite.

3. The composition of claim 2, wherein the mineral ascorbate is calcium ascorbate, magnesium ascorbate, zinc ascorbate, sodium ascorbate, or potassium ascorbate.

4. The composition of claim 1, wherein the at least one ascorbate-glucose transport enhancer is lipoic acid or corosolic acid.

5. The composition of claim 4, wherein the lipoic acid is alpha lipoic acid, ALA, r-alpha lipoic acid, RS-alpha lipoic acid, or lipoate.

6. The composition of claim 1, wherein the ascorbate is provided by at least one source selected from the group consisting of vegetables, fruit, camu fruit, alma berries, acerola cherries, rosehips, citrus fruit, extracts thereof, concentrates thereof, constituents thereof, or derivatives thereof.

7. The composition of claim 1, wherein the composition comprises ascorbate in an amount from about 0.1% by weight of actives to about 99.9% by weight of actives and at least one ascorbate-glucose transport enhancer in an amount from about 0.01% by weight of actives to about 99.0% by weight of actives.

8. The composition of claim 1, wherein the composition comprises ascorbate in an amount up to about 95% by weight of actives and at least one ascorbate-glucose transport enhancer in an amount from about 5% by weight of actives to about 10% of by weight of actives.

9. The composition of claim 1, wherein the composition is in an oral dosage form or a topical dosage form.

10. The composition of claim 9, wherein the composition is in a dosage form that is a powder, a microencapsulated powder, granules, a granulated powder, a liquid, a gel, a lotion, a cream, a spray, an emulsion, an oil, an instant beverage, a liquid beverage, a beverage mix, a capsule, a softgel capsule, a two-piece capsule, a tablet, a chewable tablet, an effervescent tablet, a pre-blended mixture of ingredients, or a blended mixture of ingredients.

11. The composition of claim 1, wherein the composition is formulated as a time release formulation, a gradual release formulation, or a fast release formulation.

12. The composition of claim 1, wherein the composition is formulated as an antioxidant vitamin formula, a multiple vitamin formula, an immune formula, or a joint formula.

13. The composition of claim 1, further comprising from about 0.1% by weight of the composition to about 90.0% by weight of the composition of an antioxidant, a threonic acid, a fruit extract, a fruit concentrate, a vegetable extract, a vegetable concentrate, a mineral, a B-Vitamin, a B-vitamin metabolite, a Carotenoid, a CoQ10, a Grapeseed extract, a Green Tea, a Lutein, a Lycopene, a Pomegranate, a Pycnogenol, a Resveratrol, a Selenium, a Zeaxanthin, a Zinc, a Copper, a Vitamin E, a Tocopherol, or a Tocotrienol.

14. The composition of claim 1, further comprising from about 1% by weight of the composition to about 95% by weight of the composition of a pepper extract, a quercetin, a rutin, a bromelain, a polyphenol, or a bioflavonoid.

15. The composition of claim 1, further comprising at least one excipient, wherein the at least one excipient is a magnesium stearate, a stearic acid, a microcrystalline cellulose, a calcium carbonate, a croscarmelose, silicon dioxide, or a starch.

16. A composition comprising ascorbate in an amount from about 0.1% by weight of actives to about 99.9% by weight of actives, and at least one ascorbate-glucose transport enhancer in an amount from about 0.01% by weight of actives to about 99.0% by weight of actives;
wherein the ascorbate is in the form of vitamin C, ascorbic acid, L-ascorbic acid, an ascorbyl ester, ascorbyl palmitate, an ascorbyl phosphate ester, a reacted or blended mineral ascorbate, dehydroascorbate, or a vitamin C metabolite; and
wherein the at least one ascorbate-glucose transport enhancer is lipoic acid or corosolic acid.

17. A method of improving the transport of acorbate into cells and tissues comprising:
providing a composition comprising ascorbate and at least one ascorbate-glucose transport enhancer, wherein the composition is in an oral dosage form or a topical dosage form;
wherein the ascorbate is in an amount from about 0.1% by weight of actives to about 99.9% by weight of actives; and
wherein the at least one ascorbate-glucose transport enhancer is in an amount from about 0.01% by weight of actives to about 99.0% by weight of actives.

18. The method of claim 17, wherein the at least one ascorbate-glucose transport enhancer is in an amount from about 5% by weight of actives to about 10% by weight of actives.

19. The method of claim 17, wherein the ascorbate is in the form of vitamin C, ascorbic acid, L-ascorbic acid, an ascorbyl ester, ascorbyl palmitate, an ascorbyl phosphate ester, a reacted or blended mineral ascorbate, dehydroascorbate, or a vitamin C metabolite, and wherein the at least one ascorbate-glucose transport enhancer is lipoic acid, corosolic acid, or threonic acid.

20. The method of claim 17, wherein the composition is in a dosage form that is a powder, a microencapsulated powder, granules, a granulated powder, a liquid, a gel, a lotion, a cream, a spray, an emulsion, an oil, an instant beverage, a liquid beverage, a beverage mix, a capsule, a softgel capsule, a two-piece capsule, a tablet, a chewable tablet, an effervescent tablet, a pre-blended mixture of ingredients, or a blended mixture of ingredients.
